(19) United States
(12) Patent Application Publication
(10) Pub. No.: US 2026/0325412 A1
(43) Pub. Date: Oct. 22, 2026

(54) COMPOSITION FOR CARING FOR THE KERATIN MATERIALS

(71) Applicant: L'OREAL, Paris (FR)

(72) Inventors: Xiaomeng WU, Shanghai (CN); Shan WU, Shanghai (CN); Xiaomin WENG, Shanghai (CN)

(73) Assignee: L'OREAL, Paris (FR)

(21) Appl. No.: 19/483,880

(22) PCT Filed: May 16, 2023
(86) PCT No.: PCT/CN2023/094507

§ 371 Date: Nov. 12, 2025

(51) Int. Cl. A61K 8/03; A61K 8/37; A61K 8/44; A61K 8/49; A61K 8/58; A61K 8/67; A61K 8/73; A61Q 1/00; A61Q 19/00; A61Q 19/02; A61Q 19/08

(52) U.S. Cl. CPC A61K 8/03; A61K 8/375; A61K 8/44; A61K 8/4933; A61K 8/585; A61K 8/671; A61K 8/731; A61Q 1/00; A61Q 19/007; A61Q 19/02; A61Q 19/08; A61K 2800/51; A61K 2800/805

(57) ABSTRACT

Provided herein is a bi-phase composition comprising: A) a continuous aqueous phase comprising a thiopyridinone compound or a derivative thereof; B) an oil phase, and C) hydrophobic particles adhered on the surface of the oil phase.

### TECHNICAL FIELD

The present invention relates to a composition, preferably for caring for keratin materials, and in particular the skin. Further, the present invention relates to use of same composition for caring for keratin materials, especially the skin.

### BACKGROUND

It has always been an ultimate goal of the cosmetic filed to deliver products with skin benefits such as anti-aging, hydration, moisturizing, whitening, cleansing, and so on.

Some oil soluble/dispersible active agents are generally more stable in un-solvated/un-dispersed state, e.g., in dry form, compared with the aqueous solution thereof, thus affecting the cosmetic effect thereof.

It is known, in particular in food and pharmaceutical field, to formulate these types of ingredients in dry form, such as beads, granules, particles, tablets, etc., or in a highly-sticky form, such as cream, rich lotion, etc. For example, traditional cosmetic compositions with high retinol content generally are prepared in cream or rich lotion, because such systems are usually easy to improve retinol stability in formula. On the other hand, however, such formulas are very rich and oily to skin.

Moreover, some water soluble/dispersible active agents are also more stable in un-solvated/un-dispersed state. For example, it is known that certain thiopyridinone compounds exhibit good depigmenting activity, even at low concentration. However, it has been discovered that a thiopyridinone compound tends to be destabilized in a composition over time, in particular when the composition including the thiopyridinone compound is maintained for a relatively long period of time under elevated temperature.

Then, when both oil and water soluble/dispersible active agents, which are more stable in dry form, are to be applied concurrently, there is a need for a new type of composition caring for the keratin materials, in particular the skin, for enhanced stability of both oil and water soluble/dispersible active agents over time.

### SUMMARY OF THE INVENTION

Therefore, in one aspect, it is preferable to provide a composition, preferably for caring for the keratin materials to dispense both oil and water soluble/dispersible active agents concurrently with desirable stability.

In another aspect, it is preferable to provide a composition, preferably for caring for the keratin materials, in particular the skin, with an improved easy usage.

One subject of the invention is thus to provide a bi-phase composition comprising:
A) a continuous aqueous phase comprising at least one thiopyridinone compound or a derivative thereof;
B) an oil phase comprising at least one oil soluble/dispersible active agent; and
C) hydrophobic particles adhered on the surface of the oil phase.

The term “hydrophobic particles adhered on the surface of the oil phase” means the bi-phase composition is stabilized by hydrophobic particles which absorb onto the interface between the aqueous phase and the oil phase to make the oil phase separate from the aqueous phase.

The thiopyridinone compound useful in the continuous aqueous phase A) is a compound of formula (I) or (I′), or their salts, their optical isomers, racemates, and/or Solvates such as hydrates and the thereof, alone or as a mixture:

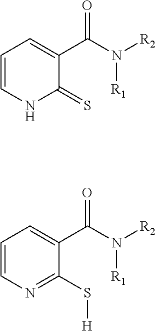

In which Formulas (I) and (I′):
R₁ denotes a radical chosen from:
a) a hydrogen atom;
b) a saturated linear C₁-C₁₀ or branched C₃-C₁₀ alkyl group optionally substituted with one or more groups, which may be identical or different, chosen from:
   i) —O—R₃
   ii) —S—R₃;
R₂ denotes a radical chosen from:
a) a hydrogen atom;
b) a saturated hydrocarbonated group linear C₁-C₁₂ or branched C₃-C₁₂ or cyclic C₃-C₈, optionally substituted with one or more groups, which may be identical or different, chosen from:
   i) —O—R₃
   ii) —S—R₃
   iii) —C(O)—O—R₃;
   iv) a C₅-C₁₂ aryl group optionally substituted with one or more hydroxyls and/or with one or more C₁-C₈ alkoxy radicals;
c) a C₅-C₁₂ aryl group optionally substituted with one or more hydroxyls and/or with one or more C₁-C₈ alkoxy radicals
R₃ denotes a radical chosen from:
a) a hydrogen atom;
b) a saturated linear C₁-C₁₀ or branched C₃-C₁₀ alkyl group;

The compound (I′) is the tautomer form of the compound (I) when a tautomeric equilibrium exists according to the following scheme:

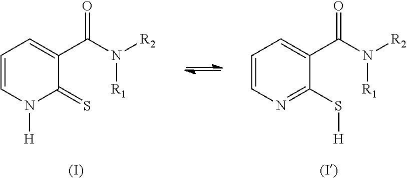

According to one embodiment of the invention R₁ represents one hydrogen atom.

According to another embodiment of the invention R₁ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably ethyl. Particularly the said alkyl group of R₁ is not substituted.

According to one embodiment of the invention R₂ represents one hydrogen atom.

According to another embodiment of the invention R₂ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl group; the said alkyl group of R₂ being not substituted.

According to another embodiment of the invention R₂ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl; the said alkyl group being substituted by one or more groups selected from i), ii), iii) and iv) as defined herein before. Preferably the said alkyl group being substituted by one or two groups selected from i), ii) and iii), more preferably by one or two groups selected from i) and iii), better substituted by one group iii) as carboxy.

Another variant for radical R₂ is that the said alkyl group being substituted by one group iv) especially substituted by one phenyl group.

According to another embodiment of the invention R₂ represents a (C₃-C₈)cycloalkyl group, preferably a (C₅-C₇)cycloalkyl group such cyclohexyl.

According to another embodiment of the invention R₂ represents C₅-C₁₂ aryl group optionally substituted with one or more hydroxyls and/or with one or more C₁-C₈ alkoxy radicals, preferably a phenyl group particularly not substituted.

According to an embodiment R₃ represents a hydrogen atom.

According to another embodiment R₃ represents a saturated linear C₁-C₁₀ or branched C₃-C₁₀ alkyl group; particularly a linear (C₁-C₆)alkyl group or a branched (C₃-C₆)alkyl group, preferably (C₁-C₄)alkyl group such as methyl group.

Another subject of the present invention is use of the composition according to the present invention for caring for the keratin materials, especially the skin. This use may manifest itself as a process for caring for the keratin materials, especially the skin.

Other characteristics and advantages of the present invention will emerge more clearly on reading the description and the examples that follow.

### EMBODIMENTS OF THE INVENTION

Throughout the description, including the claims, the term “comprising a” should be understood as being synonymous with “comprising at least one”, unless otherwise mentioned. Moreover, the expression “at least one” used in the present description is equivalent to the expression “one or more”.

Preferably, the “keratin material” according to the present invention is the skin. By “skin”, we intend all the body skin. Still preferably, the keratin material is the face, or the neck, especially the face.

Other than in the operating examples, or where otherwise indicated, all numbers expressing quantities of components and/or reaction conditions are to be understood as being modified in all instances by the term “about”, with conventionally known meaning in the art, e.g., within 10% of the indicated number (such as “about 10%” means 9%-11% and “about 2%” means 1.8%-2.2%).

In the application, unless specifically mentioned otherwise, contents, parts and percentages are expressed on a weight basis.

For the purposes of the present invention, and unless otherwise indicated:
a “saturated hydrocarbonated group linear C₁-C₁₂ or branched C₃-C₁₂” is equivalent to a “linear (C₁-C₁₂)alkyl or branched (C₃-C₁₂)alkyl group” which correspond to a saturated C₁-C₁₂ linear or branched C₃-C₁₂ hydrocarbon based group, and preferably C₁-C₁₀ linear or C₃-C₁₀ branched hydrocarbon based group, more preferably C₁-C₆ linear or C₃-C₆ branched hydrocarbon based group; preferentially, the linear or branched groups may be chosen from methyl, ethyl, propyl, isopropyl, butyl, isobutyl and tert-butyl, pentyl, hexyl, heptyl, octyl, nonyl and decyl.
More preferentially, the saturated linear or branched alkyl groups may be chosen from methyl, ethyl, propyl, isopropyl, butyl, isobutyl and tert-butyl, pentyl, hexyl, heptyl and octyl, such as methyl, ethyl, n-pentyl, n-nonyl, isobutyl.
   a saturated hydrocarbonated group cyclic C₃-C₈ is a monocyclic or bicyclic cycloalkyl group containing from 3 to 8 carbon atoms, especially is a monocyclic C₅-C₇ cycloalkyl group such as cyclohexyl group,
   an “alkoxy radical” is an alkyl-oxy radical for which the alkyl radical is a linear or branched C₁-C₁₆ and preferentially C₁-C₈ hydrocarbon-based radical;
   when the alkoxy group is optionally substituted, this implies that the alkyl group is optionally substituted as defined hereinabove;
   an “aryl” group represents a fused or non-fused monocyclic or bicyclic carbon-based group comprising from 5 to 12 carbon atoms, preferably from 6 to 10 carbon atoms, and in which at least one ring is aromatic; preferentially, the aryl radical is a phenyl, biphenyl, naphthyl, more preferably a phenyl group;
   the term “at least one” is equivalent to the term “one or more”; and
   the term “inclusive” for a range of concentrations means that the limits of that range are included in the defined range.

The salts of the compounds of formula (I), (I′), (II) or (II′) as defined hereinafter comprise the conventional non-toxic salts of said compounds, such as those formed from organic or inorganic acid or from organic or inorganic base.

As salts of the compounds of formula (I), (I′), (II) or (II′) mention may be made of:
the salts obtained by addition of the compound of formula (I) or (II) to:
   a mineral base, such as sodium hydroxide, potassium hydroxide, calcium hydroxide, ammonium hydroxide, magnesium hydroxide, lithium hydroxide, and sodium, potassium or calcium carbonate or hydrogen carbonate for example;
or
   an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made in particular of 2-amino-2-methylpropanol, ethanolamine, triethanolamine, 2-dimethylaminopropanol, 2-amino-2-(hydroxymethyl)-1,3-propanediol and 3-(dimethylamino)propylamine.

Mention may also be made of the salts of amino acids, for instance lysine, arginine, guanidine, glutamic acid and aspartic acid. Advantageously, the salts of the compounds of formula (I) or (II) (when it comprises a carboxy group) may be chosen from alkali metal or alkaline-earth metal salts such as sodium, potassium, calcium or magnesium salts and ammonium salts.
as “organic or inorganic acid salt” is more particularly chosen from salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
The acceptable solvates of the compounds described in the present invention comprise conventional solvates such as those formed during the preparation of said compounds owing to the presence of solvents. Mention may be made, by way of example, of the solvates due to the presence of water or of linear or branched alcohols, such as ethanol or isopropanol.
The optical isomers are in particular, the enantiomers and the diastereoisomers.

The present invention provides a bi-phase composition comprising:
A) a continuous aqueous phase comprising at least one thiopyridinone compound of formula (I) or (I′), or their salts, their optical isomers, racemates, and/or solvates such as hydrates and the thereof, alone or as a mixture;
B) an oil phase comprising at least one oil soluble/dispersible active agent; and
C) hydrophobic particles adhered on the surface of the oil phase.

### Continuous Aqueous Phase A)

The bi-phase composition according to the present invention comprises a continuous aqueous phase.

The continuous aqueous phase comprises water.

The continuous aqueous phase may also comprise water-miscible organic solvents (at room temperature: 20-25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers, and mixtures thereof.

The continuous aqueous phase may also comprise any water-soluble or water-dispersible compound that is compatible with an aqueous phase, such as hydrophilic gelling agents, chelating agents, antioxidant, preservatives or surfactants, and mixtures thereof.

Specific chelating agent may be particularly useful for reducing the electrostatic bonding associated with substantial presence of water. when chelating agent these ions, the ionic strength of the water decreases. For the purpose of the present invention, e.g., particularly the stability desired, trisodium ethylenediamine disuccinate can be provided as an example of chelating agent.

Similarly, specific antioxidant may be particularly useful for the purpose of the present invention, e.g., improved stability of the hiopyridinone compound of formula (I) or (I′) in aqueous phase A) and/or of the oil soluble/dispersible active agent in oil phage B). For this purpose, pentaerythrityl tetra-di-t-butyl hydroxyhydrocinnamate can be provided as an example of antioxidant.

In particular, the bi-phase composition of the present invention may comprise the continuous aqueous phase in an amount ranging from 65% to 99% by weight, especially from 70% to 90% by weight and more particularly from 75% to 85% by weight, relative to the total weight of the bi-phase composition.

### Thiopyridinone Compound

The bi-phase composition according to the invention comprises, in continuous aqueous phase A), a thiopyridinone compound of formula (I) below or tautomer (I′) below or their salts, their optical isomers, racemates, and/or solvates such as hydrates and the thereof, alone or as a mixture, as component a):

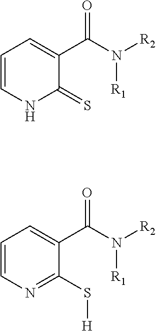

In which Formulas (I) and (I′):
R₁ denotes a radical chosen from:
a) a hydrogen atom;
b) a saturated linear C₁-C₁₀ or branched C₃-C₁₀ alkyl group optionally substituted with one or more groups, which may be identical or different, chosen from:
   i) —O—R₃
   ii) —S—R₃;
R₂ denotes a radical chosen from:
a) a hydrogen atom;
b) a saturated hydrocarbonated group linear C₁-C₁₂ or branched C₃-C₁₂ or cyclic C₃-C₈, optionally substituted with one or more groups, which may be identical or different, chosen from:
   i) —O—R₃
   ii) —S—R₃
   iii) —C(O)—O—R₃;
   iv) a C₅-C₁₂ aryl group optionally substituted with one or more hydroxyls and/or with one or more C₁-C₈ alkoxy radicals;
c) a C₅-C₁₂ aryl group optionally substituted with one or more hydroxyls and/or with one or more C₁-C₈ alkoxy radicals
R₃ denotes a radical chosen from:
a) a hydrogen atom;
b) a saturated linear C₁-C₁₀ or branched C₃-C₁₀ alkyl group;

The compound (I′) is the tautomer form of the compound (I) when a tautomeric equilibrium exists according to the following scheme:

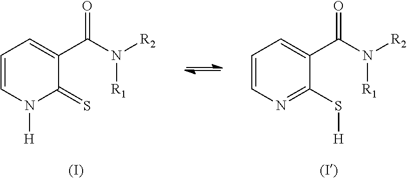

According to one embodiment of the invention R₁ represents one hydrogen atom.

According to another embodiment of the invention R₁ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably ethyl. Particularly the said alkyl group of R₁ is not substituted.

According to one embodiment of the invention R₂ represents one hydrogen atom.

According to another embodiment of the invention R₂ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl group; the said alkyl group of R₂ being not substituted.

According to another embodiment of the invention R₂ represents a linear (C₁-C₁₀)alkyl group or branched (C₃-C₁₀)alkyl group, especially a linear (C₁-C₆)alkyl group or branched (C₃-C₆)alkyl group, such as methyl, ethyl, n-pentyl, n-nonyl, isobutyl, more preferably methyl or ethyl; the said alkyl group being substituted by one or more groups selected from i), ii), iii) and iv) as defined herein before. Preferably the said alkyl group being substituted by one or two groups selected from i), ii) and iii), more preferably by one or two groups selected from i) and iii), better substituted by one group iii) as carboxy.

Another variant for radical R₂ is that the said alkyl group being substituted by one group iv) especially substituted by one phenyl group.

According to another embodiment of the invention R₂ represents a (C₃-C₈)cycloalkyl group, preferably a (C₅-C₇)cycloalkyl group such cyclohexyl.

According to another embodiment of the invention R₂ represents C₅-C₁₂ aryl group optionally substituted with one or more hydroxyls and/or with one or more C₁-C₈ alkoxy radicals, preferably a phenyl group particularly not substituted.

According to an embodiment R₃ represents a hydrogen atom.

According to another embodiment R₃ represents a saturated linear C₁-C₁₀ or branched C₃-C₁₀ alkyl group; particularly a linear (C₁-C₆)alkyl group or a branched (C₃-C₆)alkyl group, preferably (C₁-C₄)alkyl group such as methyl group.

Preferably, the compounds of formula (I) and tautomer (I′) or their salts, their optical isomers, racemates, and/or solvates such as hydrates and the thereof, alone or as a mixture;
have the following meanings:
R₁ denotes a radical chosen from:
a) a hydrogen atom;
b) a saturated linear C₁-C₆ or branched C₃-C₆ alkyl group optionally substituted with one or more groups, which may be identical or different, chosen from:
   i) —O—R₃
   ii) —S—R₃;
preferably optionally substituted with one or more groups i)
R₂ denotes a radical chosen from:
a) a hydrogen atom;
b) a saturated hydrocarbonated group linear C₁-C₁₀ or branched C₃-C₁₀ or cyclic C₃-C₈ such as C5-C6, optionally substituted with one or more groups, which may be identical or different, chosen from:
   i) —O—R₃
   ii) —SR—₃
   iii) —C(O)—O—R₃;
   iv) a phenyl group optionally substituted with one or more hydroxyls and/or with one or more C₁-C₄ alkoxy radicals such as methoxy;
preferably substituted with one or more groups selected from i) and iii), preferably iii) such as carboxy
R₃ denotes a radical chosen from:
a) a hydrogen atom;
b) a saturated linear C₁-C₆ or branched C₃-C₆ alkyl group
Preferentially, the compounds of formula (I) and tautomer (I′) or their salts, their optical isomers, racemates, and/or solvates such as hydrates and the thereof, alone or as a mixture;
have the following meanings:
R₁ denotes a radical chosen from:
a) a hydrogen atom;
b) a saturated linear C₁-C₄ or branched C₃-C₄ alkyl group optionally substituted with one or more groups, which may be identical or different, chosen from i) —OR₃, more preferably not substituted;
R₂ denotes a radical chosen from:
a) a hydrogen atom;
b) a saturated hydrocarbonated group linear C₁-C₁₀ or branched C₃-C₁₀ or cyclic C₃-C₈ as C5-C6, optionally substituted with one or more groups, which may be identical or different, chosen from:
   i) —O—R₃
   iii) —C(O)—O—R₃;
   iv) a C₅-C₁₂ aryl group optionally substituted with one or more hydroxyls and/or with one or more C₁-C₄ alkoxy radicals;
R₃ denotes a radical chosen from:
a) a hydrogen atom;
b) a saturated linear C₁-C₄ or branched C₃-C₄ alkyl group such as methyl or ethyl.

Preferentially, the compounds of formula (I) and tautomer (I′) or their salts, their optical isomers, racemates, and/or solvates such as hydrates and the thereof, alone or as a mixture;
have the following meanings:
R₁ is a hydrogen atom; and
R₂ denotes a radical chosen from:
a) a hydrogen atom;
b) a saturated hydrocarbonated group linear C₁-C₅ or branched C₃-C₅ or cyclic C₃-C₈ as C₅-C₆, substituted with one or more groups, which may be identical or different, chosen from v) —C(O)—O—R₃, preferably substituted with one group iii) —C(O)—O—R₃;
R₂ is even more preferably a saturated hydrocarbonated group linear C₁-C₄ or branched C₃-C₄ substituted with one group iii) —C(O)—OR₃.

According to another preferred embodiment, compounds of formula (I) and tautomer (I′) are selected among compounds of formula (II) and also the tautomers thereof, the salts thereof, and the optical isomers thereof, and the racemates thereof, and the solvates thereof, alone or as a mixture:

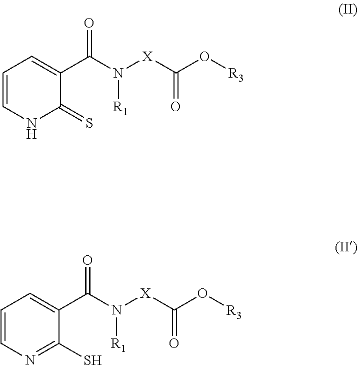

Formula (II) and (II′) Wherein R₁ and R₃ have the same meaning than for compounds of formula (I) and (I′) and X denotes an alkylene radical —(CH₂)ₙ— with n being an integer ranging inclusively from 1 to 10, preferably ranging from 1 to 6, more preferably ranging from 1 to 4, such as 1, preferably R₃ represents a hydrogen atom.

Among the compounds of formula (I), the following compounds are preferably used and their tautomer or their salts, their optical isomers, racemates, and/or solvates such as hydrates and the thereof, alone or as a mixture:

| No. | Structure | Chemical name | CAS No. |
|---|---|---|---|
| 1 | 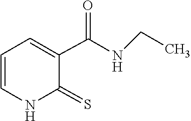 | N-ethyl-2-thioxo-1,2- dihydropyridine-3- carboxamide | 91859-75-5 |
| 2 | 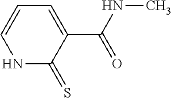 | N-methyl-2-thioxo-1,2- dihydropyridine-3- carboxamide | 91859-74-4 |
| 3 | 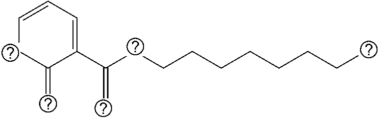 [SMILES: C=C1CC=CC=C1C(=C)CCCCCCCCC] | N-octyl-2-thioxo-1,2- dihydropyridine-3- carboxamide | 91859-77-7 |
| 4 | 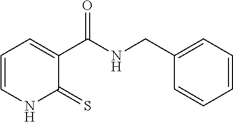 [SMILES: O=C(NCc1ccccc1)c1ccc[nH]c1=S] | N-benzyl-2-thioxo-1,2- dihydropyridine-3- carboxamide | 91859-79-9 |
| 5 | 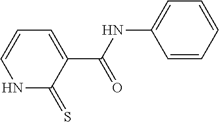 [SMILES: O=C(Nc1ccccc1)c1ccc[nH]c1=S] | N-phenyl-2-thioxo-1,2- dihydropyridine-3- carboxamide | 104857-16-1 |
| 6 | 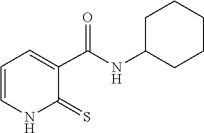 [SMILES: O=C(NC1CCCCC1)c1ccc[nH]c1=S] | N-cyclohexyl-2-thioxo- 1,2- dihydropyridine-3- carboxamide | 91859-78-8 |
| 7 | 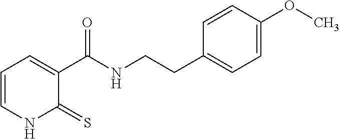 [SMILES: COc1ccc(CCNC(=O)c2ccc[nH]c2=S)cc1] | N-[2-(4-methoxyphenyl) ethyl]-2-thioxo-1,2- dihydropyridine-3- carboxamide | 923682-88-6 |
| 8 | 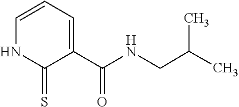 | N-(2-methylpropyl)-2- thioxo-1,2-dihydropyridine- 3-carboxamide | 1100027-79-9 |
| 9 | 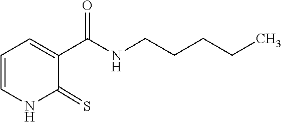 | N-pentyl-2-thioxo-1,2- dihydropyridine-3- carboxamide | 330667-57-7 |
| 10 | 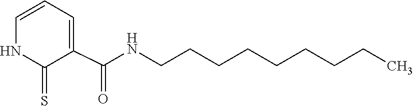 [SMILES: CCCCCCCCCNC(=O)c1ccc[nH]c1=S] | N-nonyl-2-thioxo-1,2- dihydropyridine-3- carboxamide | 1031149-44-6 |
| 11 | 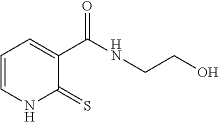 [SMILES: O=C(NCCO)c1ccc[nH]c1=S] | N-(2-hydroxyethyl)-2- thioxo-1,2-dihydropyridine- 3-carboxamide | |
| 12 | 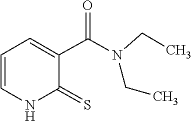 | N,N-diethyl 2-mercaptonicotinamide | |
| 13 | 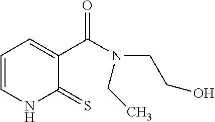 | N-ethyl-N-(2- hydroxyethyl)-2-thioxo- 1,2-dihydropyridine-3- carboxamide | |
| 14 | 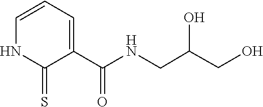 [SMILES: O=C(NCC(O)CO)c1ccc[nH]c1=S] | N-(2,3-dihydroxypropyl)-2- thioxo-1,2-dihydropyridine-3-carboxamide | |
| 15 | 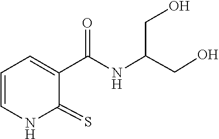 [SMILES: O=C(NC(CO)CO)c1ccc[nH]c1=S] | N-(1,3-dihydroxypropan- 2-yl)- 2-thioxo-1,2- dihydropyridine-3- carboxamide | |
| 16 | 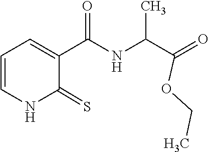 [SMILES: CCOC(=O)C(C)NC(=O)c1ccc[nH]c1=S] | Ethyl N-[(2-thioxo-1,2- dihydropyridin- 3-yl)carbonyl]alaninate | |
| 17 | 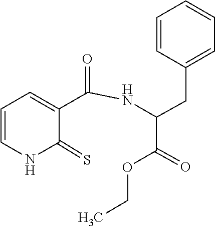 [SMILES: CCOC(=O)C(Cc1ccccc1)NC(=O)c1ccc[nH]c1=S] | Ethyl N-[(2-thioxo-1,2- dihydropyridin- 3-yl)carbonyl] phenyl alaninate | |
| 18 | 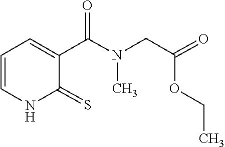 [SMILES: CCOC(=O)CN(C)C(=O)c1ccc[nH]c1=S] | Ethyl N-methyl-N-[(2-thioxo-1, 2-dihydropyridin-3-yl) carbonyl] glycinate | |
| 19 | 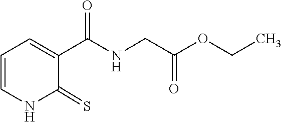 [SMILES: CCOC(=O)CNC(=O)c1ccc[nH]c1=S] | Ethyl N-[(2-thioxo-1,2- dihydropyridin-3- yl)carbonyl]glycinate | |
| 20 | 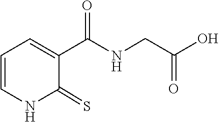 [SMILES: O=C(O)CNC(=O)c1ccc[nH]c1=S] | N-[(2-thioxo-1,2- dihydropyridin-3- yl)carbonyl]glycine | |
| 21 | 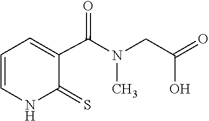 | N-methyl-N-[(2-thioxo-1,2- dihydropyridin-3-yl) carbonyl]glycine | |
| 22 | 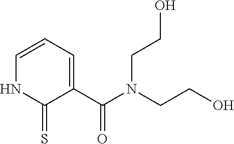 [SMILES: O=C(c1ccc[nH]c1=S)N(CCO)CCO] | N,N-bis(2-hydroxyethyl)- 2- thioxo-1,2- dihydropyridine-3- carboxamide | |
| 23 | 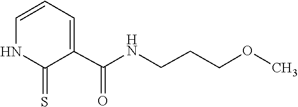 | N-(3-methoxypropyl)-2- thioxo- 1,2-dihydropyridine-3- carboxamide | |
| 24 | 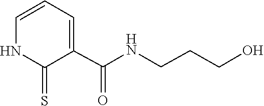 [SMILES: O=C(NCCCO)c1ccc[nH]c1=S] | N-butyl-2-thioxo-1,2- dihydropyridine-3- carboxamide | |

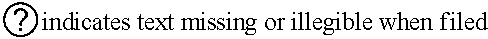

Among these compounds, the following compounds are more particularly preferred:

| No. | Structure | Chemical name | CAS No. |
|---|---|---|---|
| 1 | 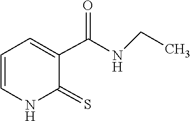 | N-ethyl-2-thioxo-1,2- dihydropyridine-3- carboxamide | 91859-75-5 |
| 2 | 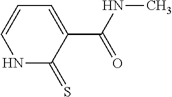 | N-methyl-2-thioxo-1,2- dihydropyridine-3-carboxamide | 91859-74-4 |
| 4 | 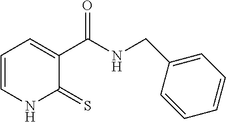 [SMILES: O=C(NCc1ccccc1)c1ccc[nH]c1=S] | N-benzyl-2-thioxo-1,2- dihydropyridine-3-carboxamide | 91859-79-9 |
| 6 | 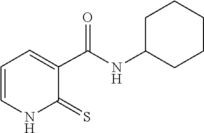 [SMILES: O=C(NC1CCCCC1)c1ccc[nH]c1=S] | N-cyclohexyl-2-thioxo-1,2- dihydropyridine-3- carboxamide | 91859-78-8 |
| 7 | 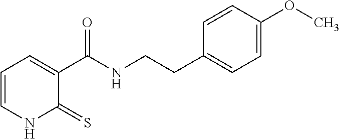 [SMILES: COc1ccc(CCNC(=O)c2ccc[nH]c2=S)cc1] | N-[2-(4-methoxyphenyl) ethyl]- 2-thioxo-1,2- dihydropyridine-3- carboxamide | 923682-88-6 |
| 9 | 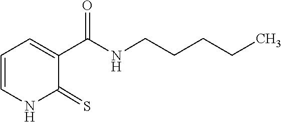 | N-pentyl-2-thioxo-1,2- dihydropyridine-3- carboxamide | 330667-57-7 |
| 11 | 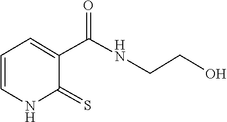 [SMILES: O=C(NCCO)c1ccc[nH]c1=S] | N-(2-hydroxyethyl)-2-thioxo- 1,2-dihydropyridine-3- carboxamide | |
| 12 | 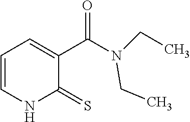 | N,N-diethyl 2-mercaptonicotinamide | |
| 14 | 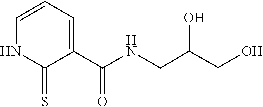 [SMILES: O=C(NCC(O)CO)c1ccc[nH]c1=S] | N-(2,3-dihydroxypropyl)-2 thioxo-1,2-dihydropyridine-3- carboxamide | |
| 15 | 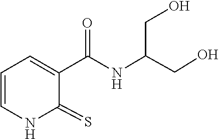 [SMILES: O=C(NC(CO)CO)c1ccc[nH]c1=S] | N-(1,3-dihydroxypropan-2- yl)- 2-thioxo-1,2-dihydropyridine-3- carboxamide | |
| 16 | 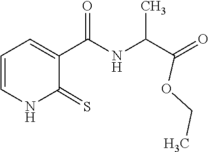 [SMILES: CCOC(=O)C(C)NC(=O)c1ccc[nH]c1=S] | Ethyl N-[(2-thioxo-1,2- dihydropyridin- 3-yl)carbonyl]alaninate | |
| 17 | 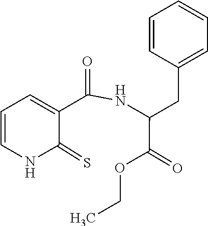 [SMILES: CCOC(=O)C(Cc1ccccc1)NC(=O)c1ccc[nH]c1=S] | Ethyl N-[(2-thioxo-1, 2-dihydropyridin-3- yl)carbonyl]phenyl alaninate | |
| 18 | 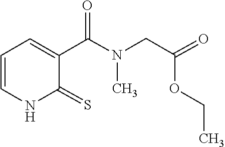 [SMILES: CCOC(=O)CN(C)C(=O)c1ccc[nH]c1=S] | Ethyl N-methyl-N-[(2-thioxo-1,2 dihydropyridin-3-yl) carbonyl] glycinate | |
| 19 | 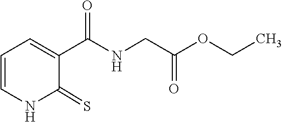 [SMILES: CCOC(=O)CNC(=O)c1ccc[nH]c1=S] | Ethyl N-[(2-thioxo-1,2- dihydropyridin-3- yl)carbonyl]glycinate | |
| 20 | 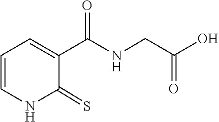 [SMILES: O=C(O)CNC(=O)c1ccc[nH]c1=S] | N-[(2-thioxo-1,2- dihydropyridin-3- yl)carbonyl]glycine | |
| 21 | 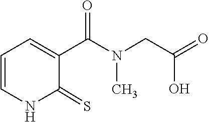 | N-methyl-N-[(2-thioxo-1,2- dihydropyridin-3-yl)carbonyl] glycine | |

More preferably, among these compounds, the following compounds are more particularly preferred:

| No. | Structure | Chemical name | CAS No. |
|---|---|---|---|
| 1 | 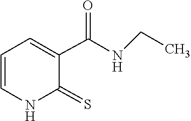 | N-ethyl-2-thioxo-1,2- dihydropyridine-3-carboxamide | 91859-75-5 |
| 9 | 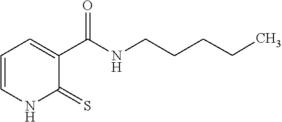 | N-pentyl-2-thioxo-1,2- dihydropyridine-3-carboxamide | 330667-57-7 |
| 16 | 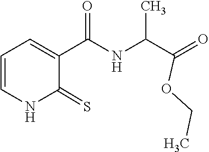 [SMILES: CCOC(=O)C(C)NC(=O)c1ccc[nH]c1=S] | Ethyl N-[(2-thioxo-1,2-dihydropyridin- 3-yl)carbonyl]alaninate | |
| 18 | 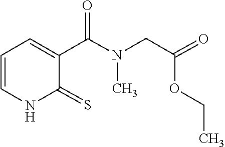 [SMILES: CCOC(=O)CN(C)C(=O)c1ccc[nH]c1=S] | Ethyl N-methyl-N-[(2-thioxo-1,2- dihydropyridin-3-yl)carbonyl] glycinate | |
| 19 | 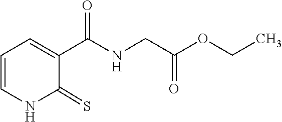 [SMILES: CCOC(=O)CNC(=O)c1ccc[nH]c1=S] | Ethyl N-[(2-thioxo-1,2-dihydropyridin- 3-yl)carbonyl]glycinate | |
| 20 | 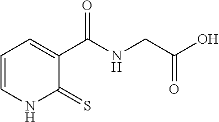 [SMILES: O=C(O)CNC(=O)c1ccc[nH]c1=S] | N-[(2-thioxo-1,2-dihydropyridin-3- yl)carbonyl]glycine | |
| 21 | 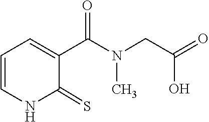 | N-methyl-N-[(2-thioxo-1,2- dihydropyridin-3-yl)carbonyl] glycine | |

Even more preferably, among these compounds, the following compounds are more particularly preferred:

| | | | CAS |
|---|---|---|---|
| No. | Structure | Chemical name | No. |
| 18 | 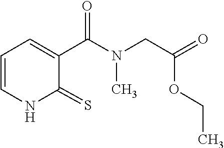 [SMILES: CCOC(=O)CN(C)C(=O)c1ccc[nH]c1=S] | Ethyl N-methyl-N- [(2-thioxo-1,2- dihydropyridin- 3-yl)carbonyl] glycinate | |
| 19 | 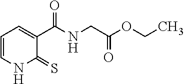 [SMILES: CCOC(=O)CNC(=O)c1ccc[nH]c1=S] | Ethyl N- [(2-thioxo-1,2- dihydropyridin- 3-yl)carbonyl] glycinate | |
| 20 | 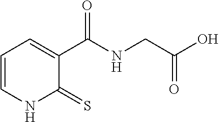 [SMILES: O=C(O)CNC(=O)c1ccc[nH]c1=S] | N-[(2-thioxo-1,2- dihydropyridin- 3-yl) carbonyl]glycine | |
| 21 | 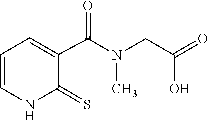 | N-methyl-N-[(2- thioxo-1,2- dihydropyridin- 3-yl)carbonyl] glycine | |

In a most preferred embodiment, the compound according to the present invention is the following:

| | | |
|---|---|---|
| 20 | 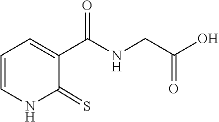 [SMILES: O=C(O)CNC(=O)c1ccc[nH]c1=S] | N-[(2-thioxo-1,2- dihydropyridin- 3-yl)carbonyl] glycine |

All compounds can be obtained by chemical method known by man skilled in the art, from commercially available reagents. For example, the synthetic method disclosed in the European patent application EP3 390 363 can be used.

The compound 20, N-[(2-thioxo-1,2-dihydropyridin-3-yl)carbonyl]glycine, may also be nominated as 3-carboxymethylaminocarbonyl-2-thiopyridinone, is a typical representative of the useful thiopyridinone compounds according to the present invention.

The thiopyridinone compound (I), (I′), (II) and/or (II′) may be present in an amount ranging for example from 0.01% to 10% by weight, preferably from 0.1% to 2% by weight, in particular from 0.5% to 1% by weight, relative to the total weight of the bi-phase composition.

### Polysaccharide

The bi-phase composition according to the invention, can comprise at least one polysaccharide.

Preferably, the continuous aqueous phase of the composition comprises at least one polysaccharide.

Preferably, the polysaccharide(s) is(are) of biotechnological origin.

In particular, these polysaccharides may, where appropriate, be chemically modified to promote its hydrophilic valency.

As examples of polysaccharides that may be used according to the invention, mention may be made especially of:
a) algal extracts such as carrageenans, alginates and agar-agar, and mixtures thereof. Examples of carrageenans that may be mentioned include Satiagum UTC30® and UTC10® from the company Degussa; an alginate that may be mentioned is the sodium alginate sold under the name Kelcosol® by the company ISP;
b) gums, such as xanthan gum, gellan gum, guar gum and nonionic derivatives thereof (hydroxypropyl guar), gum arabic, konjac gum or mannan gum, gum tragacanth, ghatti gum, karaya gum, locust bean gum, agar gum, scleroglucan gums and mixtures thereof, examples that may be mentioned include the xanthan gum sold under the name Keltrol® CG-T by the company CP Kelco, gellan gum sold under the name Kelcogel® CG LA by the company CP Kelco, guar gum sold under the name Jaguar HP 105® by the company Rhodia; mannan gum and konjac Gum® (1% glucomannan) sold by the company GfN;
c) starches, which are preferably modified, such as those derived, for example, from cereals such as wheat, corn or rice, from legumes such as white lentil, from tubers such as potato or cassava, tapioca starches; dextrins, such as dextrin extracted from corn under the name Index® from the company National Starch; Amidon de Mais B® from the company Roquette; potato feculent modified with 2-chloroethylaminodipropionic acid neutralized with sodium hydroxide, sold under the name Structure Solanace® by the company National Starch; native tapioca starch powder sold under the name Tapioca Pure® by the company National Starch;
d) pectins,
e) chitosan and derivatives thereof,
f) polyholosides comprising at least two saccharides, preferably of natural origin, and especially chosen from:
   aldoses such as
      pentoses: ribose, arabinose, xylose or apiose, for example,
      hexoses: glucose, fucose, mannose or galactose, for example,
   ketoses such as fructose,
   deoxyoses, such as rhamnose, digitoxose, cymarose or oleandrose,
   saccharide derivatives such as uronic acids, for instance mannuronic acid, guluronic acid, galacturonic acid or glycuronic acid, or itols, for instance mannitol or sorbitol.

Mention may be made in particular of the polyholosides comprising fucose, galactose and galacturonic acid units, and for example a linear sequence of α-L-fucose, α-D-galactose and galacturonic acid, for instance the biosaccharide gum-1 sold under the trade name Fucogel® 1000 PP or Fucogel® 1.5P by the company Solabia,
g) other useful polysaccharides, in particular of biotechnological origin, such as anionic polysaccharide bearing as repeating unit a tetrasaccharide composed of L-fucose, D-glucose and glucuronic acid, such as the product bearing the INCI name Biosaccharide Gum-4 sold under the reference Glycofilm 1.5P by the company Solabia,
h) and mixtures thereof.

Preferably, the polysaccharide(s) of the present invention is(are) chosen from:
gums such as xanthan gum, or gellan gum;
polyholosides comprising fucose, galactose and galacturonic acid units, for example biosaccharide gum-1
and mixtures thereof.

More preferably, the polysaccharide(s) of the present invention is(are) chosen from xanthan gum, gellan gum and mixtures thereof.

According to a preferred embodiment, the bi-phase composition of the present invention comprises from 0.0001% to 5% by weight, preferably from 0.001% to 2% by weight, more preferably from 0.01% to 1% by weight of the polysaccharides, relative to the total weight of the bi-phase composition.

### Oil Phase B)

The composition according to the invention can comprise an oil phase B).

Preferably, the oil phase comprises at least one fatty substance.

Preferably, the bi-phase composition comprises at least one fatty substance.

The term “fatty substance” means organic compounds that are insoluble in water at ordinary temperature (25° C.) and at atmospheric pressure (760 mmHg) (solubility of less than 5% by weight, preferably 1% by weight and even more preferentially 0.1% by weight). They may preferably have in their structure a sequence of at least two siloxane groups or at least one hydrocarbon-based chain comprising at least 6 carbon atoms. In addition, the fatty substances are generally soluble in organic solvents under the same temperature and pressure conditions, for instance chloroform, ethanol, benzene or decamethylcyclo pentasiloxane.

The fatty substances are especially chosen from fatty alcohols, fatty acid esters, fatty alcohol esters, fatty acid, lower alkanes, non-silicone waxes, oils, in particular animal, plant, mineral or synthetic non-silicone oils, and silicone oils.

It is recalled that, for the purposes of the invention, the fatty alcohols, fatty acid esters, fatty alcohol esters, and fatty acids more particularly contain one or more linear or branched, saturated or unsaturated hydrocarbon-based groups comprising 6 to 30 carbon atoms, which is (are) optionally substituted, in particular with one or more hydroxyl groups (in particular 1 to 4). If they are unsaturated, these compounds may comprise one to three conjugated or unconjugated carbon-carbon double bonds.

The fatty alcohols that may be used in the composition of the invention are not oxyalkylenated. They are saturated or unsaturated, linear or branched and comprise from 6 to 30 carbon atoms and more particularly from 8 to 30 carbon atoms. Mention may be made of cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol.

The esters useful are esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being more particularly greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

The composition may also comprise, as fatty ester, sugar esters and diesters of C₆-C₃₀ and preferably C₁₂-C₂₂ fatty acids. It is recalled that the term “sugar” means oxygenous hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the sugar esters or mixtures thereof described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri-, tetraesters and polyesters, and mixtures thereof.

As regards lower alkanes, these alkanes comprise from 6 to 16 carbon atoms and are linear or branched, optionally cyclic. By way of example, the alkanes may be chosen from hexane and dodecane, isoparaffins such as isohexadecane and isodecane.

Non-silicone oils that may be used in the composition of the invention can comprise particularly “hydrocarbon-based oil” (or “hydrocarbonated oil”, or “hydrocarbon oil”), which means an oil formed essentially from, or even constituted by, carbon and hydrogen atoms, and optionally oxygen and nitrogen atoms, and not containing any silicon atoms.

Examples of the non-silicone oils that may be mentioned include:
hydrocarbon-based oils of animal origin, such as perhydrosqualene;
hydrocarbon-based oils of plant origin, such as liquid fatty acid triglycerides containing from 6 to 30 carbon atoms, for instance heptanoic or octanoic acid triglycerides, or alternatively, for example, sweet almond oil, sunflower oil, corn oil, soybean oil, marrow oil, grapeseed oil, sesameseed oil, hazelnut oil, apricot oil, macadamia oil, arara oil, castor oil, avocado oil, jojoba oil, shea butter oil, and caprylic/capric acid triglycerides, for instance those sold by the company Stearineries Dubois or those sold under the names Miglyol® 810, 812 and 818 by the company Dynamit Nobel;
hydrocarbon-based oils of mineral or synthetic origin, which are linear or branched, saturated or unsaturated hydrocarbon-based oil with more than 16 carbon atoms, such as liquid paraffins, liquid petroleum jelly, polydecenes, and hydrogenated polyisobutenes such as Parleam®.

The oil phase can consist of, or consist essentially of the oil, e.g., the hydrocarbon-based oil.

Advantageously, the bi-phase composition of the present invention comprises from 0.1% to 35% by weight, preferably from 0.5% to 25% by weight, and more preferably from 1% to 20% by weight of the fatty substance, relative to the total weight of the bi-phase composition.

### Oil Soluble/Dispersible Active Agent

The oil phase B) according to the present invention comprises at least one oil soluble/dispersible active agent.

Preferably, the oil soluble/dispersible active agent is selected from the group consisting of retinoids, vitamin D, vitamin E, vitamin K, phenylethyl resorcinol or a derivative thereof, such as ester or hydrophobic modification thereof, and mixtures thereof, more preferably the oil soluble/dispersible active agent is selected from the group consisting of retinoids.

An active agent is one capable of bringing cosmetic effect to a user, especially to the skin, e.g., to face. Such cosmetic effect can be, for example, anti-aging, whitening and hydration.

Preferably, the retinoid is selected from retinoic acid, retinol (vitamin A) and its esters, such as, retinyl propionate, and retinyl acetate, or retinyl palmitate. Preferably, the retinoid is retinol.

Preferably, the oil phase B) comprises retinol as oil soluble/dispersible active agent.

The bi-phase composition of the present invention may comprise at least one oil soluble/dispersible active agent in an amount ranging from 0.01% to 2.0% by weight, preferably from 0.05% to 0.30% by weight, or preferably from 0.1% to 0.2% by weight, relative to the total weight of the bi-phase composition.

### Hydrophobic Particle C)

The bi-phase composition according to the present invention comprises hydrophobic particles. For the purpose of this invention, hydrophobic particles will adhere onto the surface of the oil phase B), so as to reduce the oxidation of the oil soluble/dispersible actives, e.g., retinoid in oil phase.

As compared, hydrophilic particles can adhere onto the surface of the aqueous phase A). Meanwhile, the thiopyridinone compound of formula (I) or a derivative thereof is contained in the aqueous phase A). Nonetheless, without being limited with any known theory, it is surprised to see that when hydrophobic particles are used, compared with either hydrophilic particles or amphiphilic particles e.g. Polysorbate 20, both the thiopyridinone compound of formula (I) or a derivative thereof in the aqueous phase A) and the oil soluble/dispersible active agent in the oil phase B) can be protected well against oxidation, so as to improve the stability thereof.

The particles may be mineral or organic, and may be in form of spherical particles or lamellar particles. The particles are preferably insoluble in the medium, e.g., solvent, oil phase, aqueous phase or the like, of the composition according to the invention, even at the melting point of the medium (e.g., about 100° C.).

In the present patent application, the term “spherical particles” means particles in the shape of sphere or at least approximate sphere, and/or a particle having an aspect ratio of about 0.9 to 1.1, e.g., about 0.95 to 1.05. For example, those skilled in the art can determine whether a particle is in the shape of sphere according to the conventional knowledge. In particular, regarding a given particle, it is within the ordinary skill of persons in the art to distinguish the shape of sphere from other shapes of plate, rod, powder, strip, irregular shapes or the like.

The term “lamellar particles” means herein particles in the shape of parallelepiped (rectangular or square surface), discoid shape (circular surface) or ellipsoid shape (oval surface), characterized by three dimensions: a length, a width and a height. In particular, regarding a given particle, it is within the ordinary skill of persons in the art to distinguish the lamellar shape from other shapes of sphere or the like.

The hydrophobic particles are different from the polysaccharide mentioned above.

According to the present invention, the hydrophobic particles may be selected from the group consisting of starches, talc, silicone resin powders, hollow hemispherical silicone particles, hydrophobic cellulose and derivatives thereof, hydrophobic silica, polyamide powders, hydrophobic pigments or any other hydrophobic particles which can be dispersed in aqueous phase.

Preferably, the hydrophobic particles are selected from the group consisting of starches, hydrophobic cellulose and derivatives thereof, hydrophobic silica, and mixtures thereof.

The starches may be preferably modified, such as those derived, for example, from cereals such as wheat, corn or rice, from legumes such as white lentil, from tubers such as potato or cassava, tapioca starches; dextrins, such as corn dextrins; Amidon de Mais B® from the company Roquette; potato feculent modified with 2-chloroethylaminodipropionic acid neutralized with sodium hydroxide, sold under the name Structure Solanace® by the company National Starch; native tapioca starch powder sold under the name Tapioca Pure® by the company National Starch.

The hydrophobic celluloses and derivatives thereof useful may be alkyl or hydroxyalkylcelluloses. Mention may be made especially of methylcelluloses, ethylcelluloses, hydroxyalkylcelluloses, ethylhydroxyethylcelluloses and carboxymethylcelluloses. Examples that may be mentioned include the hydroxyethylcellulose sold under the name Natrosol™ 250 HHR PC by the company Ashland, or under the name Cellosize™ QP 4400 H by the company Amerchol (Dow Chemical), cetylhydroxyethylcelluloses sold under the names Polysurf 67CS® and Natrosol Plus 330® from Aqualon.

According to the present invention, the hydrophobic silica may be “hydrophobically modified” silica, which means one having subjected to lipophilic modification. According to the present invention, lipophilic modification means any organo-modification capable of bringing hydrophobic or lipophilic treatment to the silica. For example, a silica can be treated with a siloxane compound, e.g., hydroxydimethylsiloxane (or other silanes, such as silazane, etc.), through which the silanol group on the surface of the silica and the silanol group of the siloxane compound are condensed. Amongst others, useful examples of “lipophilically modified” silica can be made of silica silylate available from company TOKUYAMA CORPORATION under the name of AIRLICA® TL-10 and silica dimethyl silylate available from company EVONIK DEGUSSA under the name of AEROSIL® R 972.

More preferably, the hydrophobic particles are selected from the group consisting of hydrophobic cellulose and derivatives thereof, hydrophobic silica, and mixtures thereof more preferably alkyl or hydroxyalkylcelluloses such as ethylcellulose, hydrophobic silica, and mixtures thereof.

Without being limited with any known theory, it is surprised to see that, although hydrophobic particles C) are to be added, it is further preferred that when hydrophobic celluloses or derivatives thereof are used as hydrophobic particles C), they are added in a relatively lower amount. In particular, when both the oil and water soluble/dispersible active agents are to be applied concurrently, it is believed that such a relatively lower amount of hydrophobic celluloses or derivatives thereof as hydrophobic particles C) can benefit the stabilities of the both, especially the balance between the both.

Thus, it is preferred that the bi-phase composition of the present invention comprise from 0.001% to 0.1% by weight of the hydrophobic particles C), and if used, preferably from 0.002% to 0.05% by weight, or preferably from 0.003% to 0.01% by weight of hydrophobic celluloses or derivatives thereof as the hydrophobic particles C), relative to the total weight of the bi-phase composition.

According to a preferred embodiment of the composition of the present invention, the thiopyridinone compound of formula (I) or a derivative thereof is placed in the continuous aqueous phase A) and the oil soluble/dispersible active agent is placed in the oil phase B). Then, it may be surprised to see that the stability of the entire composition is dramatically improved, compared not only with thiopyridinone compound or oil soluble/dispersible active agent alone, but also with the concurrent presence of both the thiopyridinone compound and oil soluble/dispersible active agent in a same phase, or mixed simply. In particular, as water and oil soluble/dispersible substances respectively, component a) may react with oil soluble/dispersible substances when being mixed with one another, e.g., the N-[(2-thioxo-1,2-dihydropyridin-3-yl)carbonyl]glycine (compound 20) with retinol.

### Process to Form the Composition

The process to form the bi-phase composition is not limited specifically, as long as the oil soluble/dispersible active agent can be distributed uniformly in the oil phase B), while the thiopyridinone compound of formula (I) or a derivative thereof can be distributed uniformly in the continuous aqueous phase A).

According to the present invention, the hydrophobic particles C) are added into alcohol, e.g., ethanol to obtain a mixture 1. Then, mixture 1 is added into the aqueous phase A) to obtain a mixture 2. Subsequently, mixture 2 is added into the oil phase B) to obtain the composition according to the present invention. During the additions, stirring or any other means known in the art facilitating the homogeneous mixing may be applied.

An example for the process comprises the steps of:
1). Adding the hydrophobic particles C) into alcohol, with agitating till particle dispersed, to obtain mixture 1.
2). Mixing the materials for the aqueous phase A) until all materials dissolved/dispersed, to obtain the aqueous phase A).
3). Adding mixture 1 into phase A), with stirring till uniform, to obtain mixture 2.
4). Mixing the materials for the oil phase B), with stirring till all materials dissolved, to obtain the oil phase B).
5). Adding mixture 2 into the oil phase B), with stirring for 30-40 seconds.

Following the process according to the present invention, in the composition obtained, the oil phase can be dispersed in water or under water.

The composition of the present invention can be used for a non-therapeutic process, such as a cosmetic process or method, for making up/caring for a keratin material, such as the skin, by being applied to the skin.

The invention will be further illustrated by the following Examples, which set forth particularly advantageous embodiments.

### EXAMPLES

The ingredient amounts/concentrations in the compositions/formulas described below are expressed in % by weight, relative to the total weight of each composition/formula.

The compounds are indicated in chemical name or INCI name.

| INCI Name | Trade Name | Supplier |
|---|---|---|
| RETINOL | RETINOL 10SU | BASF |
| ACETYL TRIBUTYL CITRATE | CITROFLEX A4 | VERTELLUS |
| PENTAERYTHRITYL | TINOGARD TT | BASF |
| TETRA-DI-T-BUTYL | | |
| HYDROXYHYDROCINNAMATE | | |
| TRISODIUM ETHYLENEDIAMINE | NATRLQUEST E30 | INNOSPEC ACTIVE |
| DISUCCINATE | | CHEMICALS |
| Xanthan Gum | KELTROL ®CG-T | CP KELCO |
| GELLAN GUM | KELCOGEL ® CG | CP KELCO |
| | LA | |

### Example A

The inventive compositions of Ex.1-Ex.2, as well as comparative compositions of CE.1-CE.5, were prepared as follows.

TABLE 1

| Components | | Ex. 1 | Ex. 2 | Ex. 3 | CE. 1 | CE. 3 | CE. 4 |
|---|---|---|---|---|---|---|---|
| Phase | WATER | QS | QS | QS | QS | QS | QS |
| A | ALCOHOL | 5 | 5 | 5 | 5 | 5 | 5 |
| | GELLAN GUM | 0.085 | 0.085 | 0 | 0 | 0 | 0 |
| | XANTHAN GUM | 0.085 | 0.085 | 0 | 0 | 0 | 0 |
| | N-[(2-thioxo-1,2-dihydropyridin- | 0.5 | 0.5 | 0.5 | 0.5 | 0.5 | 0.5 |
| | 3-yl)carbonyl]glycine | | | | | | |
| | (Compound 20) | | | | | | |
| | TRISODIUM | 0.2 | 0.2 | 0.2 | 0.2 | 0.2 | 0.2 |
| | ETHYLENEDIAMINE | | | | | | |
| | DISUCCINATE | | | | | | |
| | PEG-60 ALMOND | 0 | 0 | 0 | 0 | 0.5 | 0 |
| | GLYCERIDES | | | | | | |
| | POLYSORBATE 20 | | | | | | 0.3 |
| | (amphiphilic) | | | | | | |
| | CALCIUM CHLORIDE | 0.5 | 0.5 | 0.5 | 0.5 | 0.5 | 0.5 |
| Phase | ACETYL TRIBUTYL | 3 | 3 | 3 | 3 | 3 | 3 |
| B | CITRATE | | | | | | |
| | RETINOL | 0.1 | 0.1 | 0.1 | 0.1 | 0.1 | 0.1 |
| | PENTAERYTHRITYL | 0.3 | 0.3 | 0.3 | 0.3 | 0.3 | 0.3 |
| | TETRA-DI-T-BUTYL | | | | | | |
| | HYDROXYHYDROCINNAMATE | | | | | | |
| Others | ETHYLCELLULOSE | 0.003 | 0 | 0 | 0 | 0 | 0 |
| | Silica Dimethyl Silylate | 0 | 0.1 | 0.1 | 0 | 0 | 0 |

Protocol of preparation using the compositions in Table 1:
1). Mixing acetyl tributyl citrate and pentaerythrityl tetra-di-t-butyl hydroxyhydrocinnamate of phase B in container 1, stirring and heating to 80° C. to dissolve the materials completely, cooling down to 25° C., then adding retinol in container 1 to obtain a mixture 1; and
2). Mixing the materials of phase A in container 2, heating to 80° C. to dissolve the materials completely, then cooling down to 25° C.;
3). Adding others (eg. Ethyl cellulose) in container 2 to obtain a mixture 2, stirring till all materials are well mixed;
4). Adding a mixture 1 into the mixture 2, with mixing at 300 RPM for 5 min.

### II. Evaluation of the Inventive Compositions and Comparative Examples

The inventive compositions and comparative examples were evaluated using the following protocols.

### Method for Texture Transformation Evaluation

Contents of the oil soluble/dispersible active agent, i.e. retinol and N-[(2-thioxo-1,2-dihydropyridin-3-yl)carbonyl]glycine (compound 20) of all samples from the inventive compositions and comparative compositions over time were evaluated via HPLC system for TO (immediately after preparation) and T2M (two months after preparation). Specifically, after preparation, all the samples were divided into two equal parts, for which the first half parts were tested for the oil soluble/dispersible active agent, i.e. retinol and N-[(2-thioxo-1,2-dihydropyridin-3-yl)carbonyl]glycine (compound 20) contents at TO, while the second haft parts were stored under 45° C. for two months. At T2M, the second haft parts were tested for the retinol and N-[(2-thioxo-1,2-dihydropyridin-3-yl)carbonyl]glycine (compound 20) contents.

After the HPLC analyze, the accurate data of retinol content was finally got, as showed in Table 2 below.

TABLE 2

| | Ex. 1 | Ex. 2 | Ex. 3 | CE. 1 | CE. 3 | CE. 4 |
|---|---|---|---|---|---|---|
| Retinol/T0 | 0.10 | 0.10 | 0.10 | 0.10 | 0.10 | 0.10 |
| Retinol/T2M 45 °C | 0.10 | 0.09 | 0.09 | 0.05 | 0.03 | 0.02 |
| Retinol Loss compared to T0 | 0% | 10% | 10% | 50% | 70% | 80% |
| N-[(2-thioxo-1,2-dihydropyridin-3- | 0.50 | 0.50 | 0.50 | 0.50 | 0.50 | 0.50 |
| yl)carbonyl]glycine (Compound 20)/T0 | | | | | | |
| N-[(2-thioxo-1,2-dihydropyridin-3- | 0.46 | 0.45 | 0.45 | 0.41 | 0.22 | 0.19 |
| yl)carbonyl]glycine (Compound 20)/ | | | | | | |
| T2M 45 °C | | | | | | |
| N-[(2-thioxo-1,2-dihydropyridin-3- | 8% | 10% | 10% | 18% | 56% | 62% |
| yl)carbonyl]glycine (Compound 20) | | | | | | |
| Loss compared to T0 | | | | | | |

Comparing EX.3 and CE.1, it could be seen that EX.3 was formed with a special emulsion comprising hydrophobic particles and stabilizing compounds. Results showed that such a specific emulsion did protect the oil soluble/dispersible active agent, i.e., retinol in oil phase. The stability of retinol in Ex.3 was better than CE. 1.

Similarly, CE.3 represented the case of the oil soluble/dispersible active agent, i.e., retinol and N-[(2-thioxo-1,2-dihydropyridin-3-yl)carbonyl]glycine (compound 20) in a conventional emulsion system, CE.4 represented the case of the oil soluble/dispersible active agent, i.e., retinol and N-[(2-thioxo-1,2-dihydropyridin-3-yl)carbonyl]glycine (compound 20) in water-oil bi-phase (with amphiphilic particles added). the Exs.1-3 according to the present invention showed surprisingly improved stability in view of the oil soluble/dispersible active agent, i.e., retinol and/or -[(2-thioxo-1,2-dihydropyridin-3-yl)carbonyl]glycine (compound 20) compared with the comparative examples.

All publications, patents and patent applications cited in this specification are herein incorporated by reference, and for any and all purposes, as if each individual publication, patent or patent application were specifically and individually indicated to be incorporated by reference. In this case of inconsistencies, the present disclosure will prevail.

1: A bi-phase composition comprising:
A) a continuous aqueous phase comprising component a): a thiopyridinone compound of formula (I) or (I′), or their salts, their optical isomers, racemates, and/or solvates thereof, alone or as a mixture; 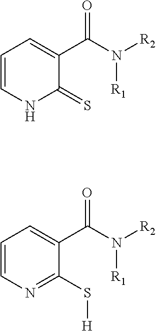
wherein
R₁ is a radical selected from:
a) a hydrogen atom; and
b) a saturated linear C₁-C₁₀ or branched C₃-C₁₀ alkyl group optionally substituted with one or more groups, each independently selected from: i) —O—R₃ and ii) —S—R₃;
R₂ is a radical selected from:
a) a hydrogen atom; and
b) a saturated hydrocarbonated linear C₁-C₁₂ group or branched C₃-C₁₂ group or cyclic C₃-C₈ group, optionally independently substituted with one or more groups, selected from:
i) —O—R₃
ii) —S—R₃
iii) —C(O)—O—R₃;
iv) a C₅-C₁₂ aryl group optionally substituted with one or more hydroxyls and/or with one or more C₁-C₈ alkoxy radicals;
c) a C₅-C₁₂ aryl group optionally substituted with one or more hydroxyls and/or with one or more C1-C8alkoxy radicals;
R₃ is a radical selected from:
a) a hydrogen atom;
b) a saturated linear C₁-C₁₀ or branched C₃-C₁₀ alkyl group;
B) an oil phase comprising at least one oil soluble/dispersible active agent; and
C) hydrophobic particles adhered on the surface of the oil phase.

2: The bi-phase composition according to claim 1, wherein:
R₁ of formula (I) and (I′) represents a hydrogen atom,
or
R₁ of formula (I) and (I′) represents a linear C₁-C₁₀ alkyl group or branched C₃-C₁₀ alkyl group.

3: The bi-phase composition according to claim 1, wherein:
R₂ of formula (I) and (I′) represents a hydrogen atom;
or
R₂ of formula (I) and (I′) represents a linear C₁-C₁₀ alkyl group or branched C₃-C₁₀ alkyl group.

4: The bi-phase composition according to claim 1, wherein:
R₂ of formula (I) and (I′) represents a linear C₁-C₁₀ alkyl group or branched C₃-C₁₀ alkyl group, the said alkyl group being substituted by one or more groups selected from i), ii), iii) and iv) as defined in claim 1.

5: The bi-phase composition according to claim 1, wherein
R₂ of formula (I) and (I′) represents a C₃-C₈ cycloalkyl group;
or
R₂ of formula (I) and (I′) represents a C₅-C₁₂ aryl group optionally substituted with one or more hydroxyls and/or with one or more C₁-C₈ alkoxy radicals.

6: The bi-phase composition according to claim 1, wherein
R₃ of formula (I) and (I′) represents a hydrogen atom;
or
R₃ of formula (I) and (I′) represents a saturated linear C₁-C₁₀ or branched C₃-C₁₀ alkyl group.

7: The bi-phase composition according to claim 1, wherein:
R₁ of formula (I) and (I′) represents a radical selected from:
a) a hydrogen atom;
b) a saturated linear C₁-C₆ or branched C₃-C₆ alkyl group optionally independently substituted with one or more groups, selected from:
i) —O—R₃, and
ii) —S—R3;
R₂ of formula (I) and (I′) represents a radical selected from:
a) a hydrogen atom;
b) a saturated hydrocarbonated linear C₁-C₁₀ group or branched C₃-C₁₀ group or cyclic C₃-C₈ group, optionally independently substituted with one or more groups selected from:
i) —O—R₃,
ii) —SR₃,
iii) —C(O)—O—R₃,
iv) a phenyl group optionally substituted with one or more hydroxyls and/or with one or more C₁-C4 alkoxy radicals;
R₃ of formula (I) and (IF) represents a radical selected from:
a) a hydrogen atom;
b) a saturated linear C₁-C₆ group or branched C₃-C₆ alkyl group.

8: The bi-phase composition according to claim 1, wherein the compound of formula (I) is selected from the group consisting of the compounds 1 to 24 and their tautomer or their salts, their optical isomers, racemates, and/or solvates such as hydrates, alone or as a mixture:

| No. | Structure | Chemical name |
|---|---|---|
| 1 | 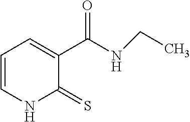 | N-ethyl-2- thioxo-1,2- dihydropyridine- 3- carboxamide |
| 2 | 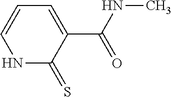 | N-methyl-2- thioxo-1,2- dihydro- pyridine-3- carboxamide |
| 3 | 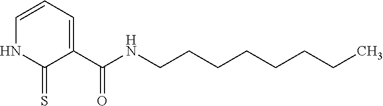 [SMILES: CCCCCCCCNC(=O)c1ccc[nH]c1=S] | N-octyl-2- thioxo-1,2- dihydropyridine- 3-carboxamide |
| 4 | 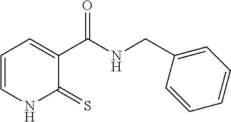 [SMILES: O=C(NCc1ccccc1)c1ccc[nH]c1=S] | N-benzyl-2- thioxo-1,2- dihydropyridine- 3-carboxamide |
| 5 | 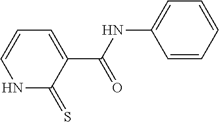 [SMILES: O=C(Nc1ccccc1)c1ccc[nH]c1=S] | N-phenyl-2- thioxo-1,2- dihydropyridine- 3-carboxamide |
| 6 | 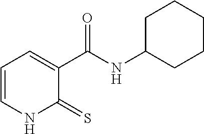 [SMILES: O=C(NC1CCCCC1)c1ccc[nH]c1=S] | N-cyclohexyl- 2-thioxo-1,2- dihydropyridine- 3-carboxamide |
| 7 | 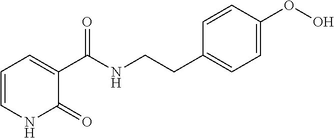 [SMILES: O=C(NCCc1ccc(OO)cc1)c1ccc[nH]c1=O] | N-[2-(4-methoxy- phenyl)ethyl]-2- thioxo-1,2- dihydropyridine- 3-carboxamide |
| 8 | 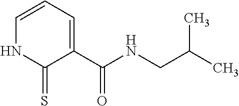 | N-(2-methyl- propyl)-2-thioxo- 1,2-dihydro- pyridine-3- carboxamide |
| 9 | 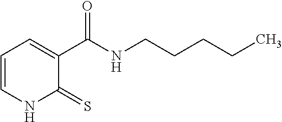 | N-pentyl-2- thioxo-1,2- dihydropyridine- 3-carboxamide |
| 10 | 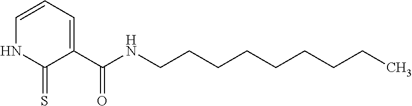 [SMILES: CCCCCCCCCNC(=O)c1ccc[nH]c1=S] | N-nonyl-2- thioxo-1,2- dihydropyridine- 3-carboxamide |
| 11 | 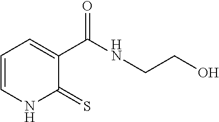 [SMILES: O=C(NCCO)c1ccc[nH]c1=S] | N-(2-hydroxy- ethyl)-2-thioxo- 1,2- dihydro- pyridine-3- carboxamide |
| 12 | 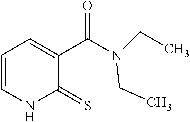 | N,N-diethyl 2- mercaptonico- tinamide |
| 13 | 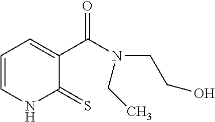 | N-ethyl-N-(2- hydroxyethyl)- 2-thioxo-1,2- dihydropyridine- 3-carboxamide |
| 14 | 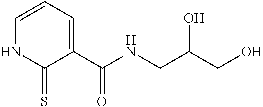 [SMILES: O=C(NCC(O)CO)c1ccc[nH]c1=S] | N-(2,3-dihydroxy- propyl)-2-thioxo- 1,2- dihydropyridine- 3-carboxamide |
| 15 | 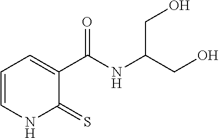 [SMILES: O=C(NC(CO)CO)c1ccc[nH]c1=S] | N-(1,3-dihydroxy- propan-2-yl)-2- thioxo-1,2- dihydropyridine- 3-carboxamide |
| 16 | 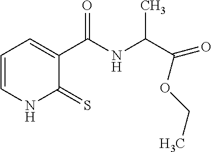 [SMILES: CCOC(=O)C(C)NC(=O)c1ccc[nH]c1=S] | Ethyl N-[(2-thioxo-1,2- dihydropyridin- 3-yl)carbonyl] alaninate |
| 17 | 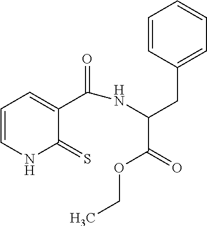 [SMILES: CCOC(=O)C(Cc1ccccc1)NC(=O)c1ccc[nH]c1=S] | Ethyl N-[(2-thioxo-1,2- dihydropyridin- 3-yl) carbonyl]phenyl alaninate |
| 18 | 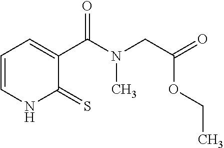 [SMILES: CCOC(=O)CN(C)C(=O)c1ccc[nH]c1=S] | Ethyl N-methyl-N-[(2- thioxo-1,2- dihydropyridin- 3-yl)carbonyl] glycinate |
| 19 | 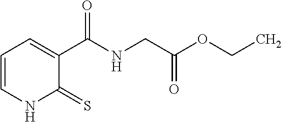 [SMILES: CCOC(=O)CNC(=O)c1ccc[nH]c1=S] | Ethyl N-[(2-thioxo-1,2- dihydropyridin- 3-yl) carbonyl]glycinate |
| 20 | 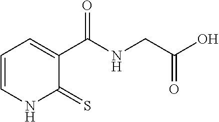 [SMILES: O=C(O)CNC(=O)c1ccc[nH]c1=S] | N-[(2-thioxo-1,2- dihydropyridin- 3-yl) carbonyl]glycine |

9: The bi-phase composition according to claim 1, wherein the thiopyridinone of compound (I) or (I′), as component a), is present in an amount ranging from 0.01% to 10% by weight, relative to the total weight of the bi-phase composition.

10: The bi-phase composition according to claim 1, wherein the oil soluble/dispersible active agent is selected from the group consisting of retinoids, vitamin D, vitamin E, vitamin K, phenylethyl resorcinol or a derivative thereof, or hydrophobic modification thereof, and mixtures thereof.

11: The bi-phase composition according to claim 10, wherein the oil soluble/dispersible active agent is a retinoid which is selected from retinoic acid, retinol (vitamin A) and its esters.

12: The bi-phase composition according to claim 1, wherein the oil soluble/dispersible active agent is present in an amount ranging from 0.01% to 2.0% by weight relative to the total weight of the bi-phase composition.

13: The bi-phase composition according to claim 1, wherein the hydrophobic particles C) are selected from the group consisting of starches, talcs, silicone resin powders, hollow hemispherical silicone particles, hydrophobic celluloses and derivatives thereof.

14: The bi-phase composition according to claim 1, wherein the hydrophobic particles C) are present in an amount ranging from 0.001%-0.1% by weight relative to the total weight of the bi-phase composition.

15: A method for caring for keratin materials, comprising application of the bi-phase composition according to claim 1 to the keratin material.

16. (canceled)
